(19) United States
(12) Patent Application Publication
(10) Pub. No.: US 2016/0000727 A1
(43) Pub. Date: Jan. 7, 2016

(54) BETA CAROTENE PREPARATION

(71) Applicant: SANOCHEMIA AG, Zug (CH)

(72) Inventor: Werner J. FRANTSITS, Wien (AT)

(73) Assignee: SANOCHEMIA AG, Zug (CH)

(21) Appl. No.: 14/765,473

(22) PCT Filed: Aug. 22, 2014
(86) PCT No.: PCT/EP2014/002310

§ 371 Date: Aug. 3, 2015

(30) Foreign Application Priority Data
Sep. 2, 2013 (AT) A 680/2013

(51) Int. Cl. A61K 31/015; A61K 31/198

(52) U.S. Cl. CPC A61K 31/015; A61K 31/198

(57) ABSTRACT

An aqueous emulsion for use in veterinary medicine which contains beta carotene and can be administered parenterally from a sterile multi-dose container contains acetylcysteine in an amount of between 1 and 8 wt.-% as a substance which stabilizes the emulsion and protects preservatives and beta carotene from being decomposed. The beta carotene-containing emulsion can additionally contain at least one antioxidant and at least one preservative. As the antioxidant, ascorbyl palmitate or alpha-tocopherol can be added. The emulsion can further contain a solubilizer, for example isopropyl myristate or Solutol® HS 15.

The invention relates to a preparation of beta carotene, in particular an aqueous emulsion of beta carotene, which is suitable for parenteral administration (DE 196 09 477 A1).

An aqueous preparation of beta carotene, which can be used in veterinary medicine, is known from EP 1 016 404 A1 (AT 408 186 B). This aqueous preparation of beta carotene, in which beta carotene is present as a micellar solution (microemulsion), contains an anionic or non-ionic solubilizer, for example polyoxyethylene-660-hydroxystearate and/or isopropyl myristate. The preparation, which contains, for example, 0.1 to 10% (w/v) beta carotene, can furthermore contain antioxidants and at least one preservative.

An injection solution from multi-dose containers, which solution can be used in veterinary medicine and contains beta carotene, is marketed by Alvetra and Werfft in Vienna under the trade name “Carofertin.” Per milliliter of injection solution, the known injection solution contains 10.0 mg of beta carotene, 10.0 mg of benzyl alcohol, 0.12 mg of ascorbyl palmitate, 0.10 mg of alpha-tocopherol, macrogol-15-hydroxystearate (Solutol HS 15), isopropyl myristate, and water for injection purposes.

It is known that beta carotene acts on the organism in two different ways. On the one hand, it is converted as a provitamin into vitamin A and exerts its action via this metabolic step; on the other hand, beta carotene itself actively intervenes in metabolism. Beta carotene has a stabilizing effect on the corpora lutea of the ovaries, ensures stimulation of follicular growth, and has a protective and anti-inflammatory influence on the endometrium.

Parenteral administration of beta carotene in pigs one week before mating increases the litter size. This effect is especially clearly pronounced in older sows.

Adequately supplying dams with beta carotene in addition brings about a clear increase in immunity of the newborns, with reduced susceptibility to intestinal infections and thus small losses in the breeding phase.

It is problematic in the known emulsions of beta carotene that the preservation system is stable for only a relatively short time, namely 12 to at most 15 months, in the closed state of the container, and after the injection solution is first opened, the system breaks down within just a few days, whereby the decomposition of the preservative and thus that of the active ingredient exceeds the allowed limits.

Since the usability of the known beta carotene emulsions in veterinary medicine was limited to such an extent, there was a problem with too short a shelf life, which in veterinary practice leads to frequent discarding of just-opened injection flasks and thus to unnecessary losses.

In EP 1 016 404 A1 and AT 408 186 B, it is mentioned that emulsions of beta carotene are problematic, since they have an only slight stability relative to spontaneously-occurring phase separation. Furthermore, the resistance of beta carotene to oxidation by oxygen that is contained in the air is poor in emulsions.

DE 196 09 477 A1 describes aqueous solubilizates, which are suitable for parenteral administration and which contain at least one carotenoid, at least one water-insoluble vitamin, and a non-ionic emulsifier. In a preferred embodiment, in addition to the carotenoid, the formulation contains (a) tocopherol (ester), ascorbic acid, as well as optionally N-acetylcysteine. As a non-ionic emulsifier, polyoxyethylene-12-hydroxystearate is mentioned. The formulation that is known from DE 196 09 477 A1 is intended for immediate consumption and is unsuitable for delivery in a multi-dose container.

DE 198 19 616 A1 discloses a composition for treatment/prophylaxis of inflammatory skin diseases, which contains N-acetylcysteine and at least one additional antioxidant, selected from the group of ascorbic acid, α-tocopherol, β-carotene and/or derivatives of the same. The composition can be administered parenterally and is suitable only for delivery in a single-dose container.

The subject matter of DE 197 47 546 A1 is the use of systemically-administered water-soluble antioxidants (e.g., ascorbate, N-acetylcysteine) together with lipid-soluble antioxidants (e.g., carotenoids, tocopherol) for treatment of inflammatory dermatoses. This preparation is suitable only for delivery in a single-dose container.

The object of the invention is to make available an aqueous preparation of beta carotene that is suitable in particular for parenteral administration, primarily in veterinary medicine, on the one hand, and a method for the production of the same, on the other hand, whereby the preparation can be delivered in multi-dose containers.

This object is achieved according to the invention with a preparation with the features of claim 1.

Insofar as the production method is concerned, the object underlying the invention is achieved with the features mentioned in the independent claim that is aimed at the production method.

The invention is based on the surprising finding that the addition of acetylcysteine to emulsions that contain beta carotene exerts a strongly stabilizing influence on the preservation system and thus improves the stability of the emulsion when the preparation contains an antioxidant and benzyl alcohol as a preservative.

Acetylcysteine (L-α-acetamido-β-mercaptopropionic acid) is a substance that is known in the art, in particular a pharmaceutical substance, which is used as an expectorant in the case of respiratory disorders and as an antidote in the case of paracetamol poisoning.

Acetylcysteine is also used in nephrology, in infectious diseases, and in psychiatry. Acetylcysteine is known neither as a stabilizer nor as a preservative or solubilizer, so that the above-described effect of the stabilization of beta-carotene-containing emulsions is surprising.

Beta carotene emulsions according to the invention, which are suitable for parenteral administration, are stable in unopened containers (e.g., multi-dose containers) for years and for at least 8 weeks after they are first opened, which makes it possible to use the emulsion to increase fertility, in particular in cows and pigs, as well as for treating disorders in dog fertility systems.

Within the framework of the invention, consideration is given in particular to the fact that the content of acetylcysteine lies in the range from 1% by weight to 8% by weight, for example at 3% by weight, relative to the emulsion.

Other possible components of the aqueous emulsion of beta carotene according to the invention are, in addition to beta carotene, in particular ascorbyl palmitate, alpha-tocopherol, benzyl alcohol, solubilizers such as isopropyl myristate, at least a non-ionic solubilizer, such as Macrogol-15-hydroxystearat (Solutol HS15), and water. An aqueous emulsion of beta carotene according to the invention preferably contains beta carotene per 1,000 g of emulsion in amounts of 5 to 15 g, in particular in an amount of 10 g per 1,000 g of emulsion.

The active ingredients ascorbyl palmitate and alpha-tocopherol that are optionally used as antioxidants are used in amounts of 0.05 to 0.50 g, preferably 0.12 g (ascorbyl palmitate) and 0.05 to 0.20 g, preferably 0.10 g (alpha-tocopherol) per 1,000 g of emulsion.

The isopropyl myristate that is optionally added as additional solubilizer can be contained in amounts of 70 to 90 g, in particular 84 g per 1,000 g of emulsion.

The acetylcysteine (preservative) that stabilizes the emulsion and that protects beta carotene from being decomposed can be present in amounts of between 1 and 8 g, in particular 3 g, per 1,000 g of emulsion.

In the method according to the invention for the production of the aqueous emulsion of beta carotene according to the invention, the step of adding isopropyl myristate to the solubilizer (e.g., Solutol) that is, for example, molten and preferably non-ionic is carried out at a temperature of between 25° Celsius and 40° Celsius, preferably at 30° Celsius. Below, additional details of the invention are described based on preferred embodiments.

### EXAMPLE 1

An aqueous emulsion of beta carotene, which is suitable for parenteral administration in veterinary medicine, has the following composition:

10.0 g of beta carotene, 0.12 g of ascorbyl palmitate, 0.10 g of alpha-tocopherol, 10.0 g of benzyl alcohol, 84.0 g of isopropyl myristate, 3.0 g of acetylcysteine, 182.0 g of Solutol HS 15, and 711.0 g of water. Combined: 1,000.0 g.

### EXAMPLE 2

An aqueous emulsion of beta carotene, which is suitable for parenteral administration in veterinary medicine, has the following composition:

15.0 g of beta carotene, 0.18 g of ascorbyl palmitate, 0.22 g of α-tocopherol, 90.0 g of isopropyl myristate, 4.5 g of acetylcysteine, 200.0 g of Solutol HS 15, and 690.1 g of water.

Combined: 1,000.0 g.

### EXAMPLE 3

An aqueous emulsion of beta carotene, which is suitable for parenteral administration in veterinary medicine, has the following composition:

7.5 g of beta carotene, 0.15 g of ascorbyl palmitate, 0.10 g of α-tocopherol, 75.0 g of isopropyl myristate, 2.0 g of acetylcysteine, 175.0 g of Solutol HS 15, and 740.25 g of water.

Combined: 1,000.0 g.

### EXAMPLE 4

An aqueous emulsion of beta carotene, which is suitable for parenteral administration in veterinary medicine, has the following composition:

5.0 g of beta carotene, 0.10 g of ascorbyl palmitate, 15.0 g of α-tocopherol, 80.0 g of isopropyl myristate, 6.0 g of acetylcysteine, 180.0 g of Solutol HS 15, and 713.9 g of water.

Combined: 1,000.0 g.

### EXAMPLE 5

The aqueous beta carotene emulsions described in Examples 1 to 4 can be produced as follows:

As a non-ionic solubilizer, Solutol HS 15 (2-hydroxyethyl-12-hydroxyoctadecanoate, macrogol-15-hydroxystearate) is heated to 60° Celsius in the batch tank, melts, and is then runny.

Liquid isopropyl myristate is added to the molten Solutol HS 15. The mixture is heated to 130° Celsius with moderate stirring.

Beta carotene is slowly added to the thus obtained solution at elevated temperature of the solution and with moderate stirring, and stirring is continued until a dark red, clear solution is present. In this case, the procedure can be performed under nitrogen atmosphere.

During this operating step (addition of the oily mixture of beta carotene and subsequent stirring), a temperature of 90° Celsius is maintained. In this case, the procedure can be performed under nitrogen atmosphere.

In a separate container, ascorbyl palmitate and alpha-tocopherol are dissolved in benzyl alcohol at room temperature.

After the thus obtained beta-carotene-containing emulsion has been slowly cooled to 50° Celsius, the previously obtained solution of ascorbyl palmitate, alpha-tocopherol in benzyl alcohol is introduced into the emulsion while being stirred. Also, in this step, the procedure is preferably performed under nitrogen atmosphere.

As the next step, acetylcysteine is dissolved in water at a temperature of 30° Celsius.

As soon as the previously obtained beta carotene-containing emulsion is cooled to 30° Celsius, the acetylcysteine solution, preferably under nitrogen atmosphere, is stirred slowly into the emulsion and stirred moderately until a dark red, clear emulsion is present.

In the tables below, the results of stability tests are presented.

TABLE I
Beta-Carotene Preparation According to AT 408 186 B1:

| | Contents After | | |
|---|---|---|---|
| | Production | After 6 Months | After 12 Months |
| Beta Carotene (All- | 99.5-101% | 97.5-99% | 96-97% |
| Trans + Cis) | | | |
| Benzyl Alcohol | 100-101.5% | 78-86% | 62-65% |
| Ascorbyl Palmitate | 99-101% | 69-74% | 58-64% |
| Tocopherol | 99-101% | 98-99% | 95-97% |

TABLE II
Beta-Carotene Preparation According to the Invention: Example 1:

| | Contents After | | |
|---|---|---|---|
| | Production | After 6 Months | After 12 Months |
| Beta Carotene (All- | 99.5-101% | 99.5-100.5% | 99-100% |
| Trans + Cis) | | | |
| Benzyl Alcohol | 100-101.5% | 99.5-100% | 98-99.5% |
| Ascorbyl Palmitate | 99-101% | 98-100% | 94-97% |
| Tocopherol | 99-101% | 99-100.5% | 97-99.5% |

TABLE III
Beta-Carotene Preparation According to the Invention: Example 2:

| | Contents After | | |
|---|---|---|---|
| | Production | After 6 Months | After 12 Months |
| Beta Carotene (All- | 99.7-100.6% | 99.4-99.9% | 98.4-99.1% |
| Trans + Cis) | | | |
| Benzyl Alcohol | 100.4-100.8% | 98.8-99.4% | 97.9-98.2% |
| Ascorbyl Palmitate | 99.6-100.7% | 97.6-99.3% | 93.4-96.2% |
| Tocopherol | 100.2-101.3% | 97.8-99.4% | 94.1-95.7% |

TABLE VI [SIC]
Beta-Carotene Preparation According to the Invention: Example 3:

| | Contents After | | |
|---|---|---|---|
| | Production | After 6 Months | After 12 Months |
| Beta Carotene (All- | 100.6-101.3% | 99.1-100.4% | 98.6-99.6% |
| Trans + Cis) | | | |
| Benzyl Alcohol | 100.3-100.8% | 97.1-97.9% | 95.6-95.9% |
| Ascorbyl Palmitate | 100.0-100.7% | 95.5-96.3% | 93.0-93.8% |
| Tocopherol | 99.7-100.8% | 96.9-98.0% | 93.1-93.6% |

TABLE V
Beta-Carotene Preparation According to the Invention: Example 4:

| | Contents After | | |
|---|---|---|---|
| | Production | After 6 Months | After 12 Months |
| Beta Carotene (All- | 99.9-100.4% | 98.8-99.6% | 98.8-99.2% |
| Trans + Cis) | | | |
| Benzyl Alcohol | 100.4-101.2% | 96.9-97.9% | 94.7-94.9% |
| Ascorbyl Palmitate | 99.7-100.4% | 97.8-99.3% | 93.3-93.9% |
| Tocopherol | 100.5-101.1% | 97.5-98.1% | 93.6-94.2% |

The contents of acetylcysteine that are not mentioned in Tables II to V correspond to the contents mentioned in Examples 1 to 4.

In the stability tests, the sample sizes per test batch of 85 liters, decanted into 100 ml cutoff flasks (unopened), were used.

Since, in veterinary medicine, room temperature is desired as a storage condition, the following storage conditions were maintained for the stability test:

25° Celsius at 60% atmospheric humidity.

From the stability data of a known beta carotene preparation presented above in Table I and stability data of beta carotene preparations according to the invention presented in Tables II to V, it is evident that the addition of acetylcysteine protects not only beta carotene but also the portion of preservatives (the “preservation system”) contained in the preparation from being decomposed, i.e., increases the stability of the preparation.

In summary, an embodiment of the invention can be described as follows:

A beta-carotene-containing aqueous emulsion, which can be used in veterinary medicine, for parenteral administration from a sterile multi-dose container contains acetylcysteine in amounts of between 1 and 8% by weight as the emulsion-stabilizing substance as well as a preservative and substance that protects beta carotene from being decomposed. In addition, the beta-carotene-containing emulsion can contain at least one antioxidant and at least one preservative. As antioxidants, ascorbyl palmitate or alpha-tocopherol can be added. Furthermore, the emulsion can contain a solubilizer, for example isopropyl myristate or Solutol HS 15.

1-14. (canceled)

15. Preparation of beta carotene in an aqueous medium, whereby the preparation is present as an emulsion for parenteral administration and contains acetylcysteine, characterized in that the preparation contains at least one antioxidant, in particular ascorbyl palmitate and/or alpha-tocopherol, as well as benzyl alcohol as a preservative.

16. The preparation according to claim 15, wherein acetylcysteine is present relative to the emulsion in amounts of between 1 and 8% by weight, preferably 2 to 5% by weight, and especially preferably 3% by weight.

17. The preparation according to claim 15, wherein the emulsion contains a solubilizer, in particular isopropyl myristate.

18. The preparation according to claim 15, wherein the emulsion contains at least one non-ionic solubilizer, in particular hydroxyethyl-12-hydroxyoctadecanoate and macrogol-15-hydroxystearate (Solutol HS 15).

19. Method for the production of an emulsion according to claim 17, characterized by the following process steps:
a) Heating the non-ionic solubilizer, in particular the hydroxyethyl-12-hydroxyoctadecanoate, in order to melt the non-ionic solubilizer, and adding isopropyl myristate to the molten solubilizer,
b) Stirring beta carotene into the solution obtained in step a),
c) Introducing the beta-carotene-containing mixture that is obtained in step b) into water while being stirred,
d) Stirring the mixture that is obtained in step c) until a dark red, clear emulsion is present,
e) Dissolving at least one antioxidant, in particular in an organic solvent, which is simultaneously used as a preservative, such as benzyl alcohol,
f) Stirring the solution of the antioxidant, obtained in step e), into the beta-carotene-containing emulsion that is obtained in step d), and
g) Adding acetylcysteine into the emulsion that is obtained in step f).

20. The method according to claim 19, wherein the non-ionic solubilizer is heated to a temperature of between 135° Celsius and 140° Celsius before isopropyl myristate is added.

21. The method according to claim 20, wherein isopropyl myristate is added to the molten solubilizer at a temperature of between 125 and 140° Celsius, in particular at 130° Celsius.

22. The method according to claim 20, wherein the mixture that contains beta carotene, molten non-ionic solubilizer and isopropyl myristate that is obtained in step b) is introduced into heated water and stirred until a dark red, clear emulsion is present.

23. The method according to claim 19, wherein it is stirred in step b) at a temperature of 90° Celsius.

24. The method according to claim 19, wherein the beta-carotene-containing emulsion that is obtained in step d) is added to a solution of at least one antioxidant at a temperature of approximately 50°.

25. The method according to claim 24, wherein the emulsion that is mixed with antioxidant is mixed in at a temperature of 30° Celsius with acetylcysteine, in particular acetylcysteine that is dissolved in water.

26. The method according to claim 19, wherein the process steps, with the exception of the first process step a), are carried out under nitrogen atmosphere.

27. The method according to claim 21, wherein the mixture that contains beta carotene, molten non-ionic solubilizer and isopropyl myristate that is obtained in step b) is introduced into heated water and stirred until a dark red, clear emulsion is present.

28. The method according to claim 20, wherein it is stirred in step b) at a temperature of 90° Celsius.

29. The method according to claim 20, wherein the beta-carotene-containing emulsion that is obtained in step d) is added to a solution of at least one antioxidant at a temperature of approximately 50°.

30. The method according to claim 20, wherein the process steps, with the exception of the first process step a), are carried out under nitrogen atmosphere.
